(19) United States
(12) Patent Application Publication
(10) Pub. No.: US 2002/0155438 A1
(43) Pub. Date: Oct. 24, 2002

(54) METHOD FOR DETERMINING NUCLEOTIDE SEQUENCES USING ARBITRARY PRIMERS AND LOW STRINGENCY

(76) Inventors: ANDREW JOHN GEORGE SIMPSON, SAO PAULO (BR); EMMANUEL DIAS NETO, SAO PAULO (BR); RICARDO RENZO BRENTANI, SAO PAULO (BR)

Correspondence Address: FULBRIGHT & JAWORSKI, LLP, 666 FIFTH AVE, NEW YORK, NY, 10103-3198, US

(21) Appl. No.: 09/406,117

(22) Filed: Sep. 27, 1999

Related U.S. Application Data
(63) Continuation-in-part of application No. 09/196,716, filed on Nov. 20, 1998.

(51) Int. Cl. C12Q 1/68; C07H 21/02; C07H 21/04

(52) U.S. Cl. 435/6; 536/23.1

(57) ABSTRACT

The invention involves a method for obtaining sequence information from nucleic acid molecules, such as cDNA. The method involves the use of arbitrary primers, and low stringency conditions. Rather than providing information from the termini of nucleic molecules, the method provides information on the more interesting and relevent internal portions of nucleic acid molecules. The method shows how to secure information on ORFs, and how to prepare contig sequences from any source.

### RELATED APPLICATIONS

This application is a continuation in part of Application Ser. No. 09/196,716, filed on Nov. 20, 1998, the disclosure of which is incorporated by reference in its entirety.

### FIELD OF THE INVENTION

The invention relates to methods for determining the sequences of nucleic acid molecules. More particularly, it relates to a method for preferentially sequencing internal portions of nucleic acid molecules, such as those portions referred to as open reading frames, or “ORFs”. The method is such that one can essentially eliminate sequencing of non-coding portions. Preferentially, the method is applied to complementary DNA, or “cDNA” obtained from eukaryotes. The method is applicable to all organisms, eukaryotic organisms in particular, be they single cell or complex. All nucleic acid molecules including plant and animal molecules can be studied with this method. Repeated application of the method permits the sequencing of essentially the entire coding component of an organism, regardless of the complexity of the genome under consideration. Application of the method has led to the identification of hundreds of previously unknown nucleic acid molecules. Further application of the method permits the construction of “contigs” or constructs of sequenced nucleic acid molecules. Application of the method also allows one to assign previously identified nucleotide sequences to internal regions of genes.

### BACKGROUND AND PRIOR ART

The area of nucleic acid research has seen tremendous advances in knowledge and understanding in the recent past. One of the goals in the field has been the determination of the sequence ofthe entire chromosomal component, or “genome” of organisms. This has been achieved for several non-nucleated organisms (prokaryotes), and of one organism with a nucleus, a “eukaryote”. Eukaryotes have much more complex genomes than prokaryotes, for reasons which will be discussed infra.

The interest in sequencing entire genomes of organisms has been explained in detail in both technical and non-technical publications, and need not be repeated here. See, for example Venter, et al, “Shotgun Sequencing of The Human Genome”, Science 280:1640-1642 (1998), Pennisi, “A Planned Boost for Genome Sequencing, But the Plan Is in Flux”, Science 281: 148-149 (1998).

Various approaches to what is a large, and complex project have been advanced. For example, the so-called “Shotgun” approach, developed by Venter et al, is very well known. In this approach, genomic DNA is cleaved into very small pieces, and these pieces are then sequenced. The approach is repeated, and after an undefined number of repeats, sequences are aligned to permit, at least in theory, a determination of the complete genomic sequence.

This approach has been used by Venter et al on prokaryotes, and it has been proposed for use on more complex eukaryotes, such as humans. The proposed approach to eukaryotes is not without drawbacks and criticism, however. A sizable portion of the scientific community is ofthe view that the resulting information will be riddled with gaps. The human genome, in contrast to prokaryotic genomes is characterized by a large number of repetitive sequences. It is felt by many that the overlapping of repetitive sequences could lead to incorrect alignment of the larger fragments from which they are derived.

A second approach, which has found more widespread acceptance, is to cleave the genome into relatively large fragments, and then to “map” the larger, non-sequenced fragments to show overlap prior to sequencing the material. After this overlapping, which results in a physical map of the genome, the segments are fragmented, and sequenced. While this approach should, in theory, eliminate the gaps in the sequence, it is time consuming and costly. Further, both of these approaches suffer from a fundamental drawback, as will all approaches which begin with eukaryotic genomic DNA, as will now be explained.

Eukaryotic DNA consists of both “coding” and “non-coding” DNA. For purposes of this invention, only coding DNA is under consideration, as it is this material which is transcribed and then translated into proteins. This coding DNA is sometimes referred to as “open reading frames” or “ORFs”, and this terminology will be used hereafter.

As compared to prokaryotes, eukaryotic DNA has a much more complex structure. Genes generally consist of a non-coding, regulatory portion ofhundreds of nucleotides followed by coding regions (“exons”), separated by non-coding regions (“introns”). When DNA is transcribed into messenger RNA, or mRNA, and then translated into protein, it is only these exons which are of interest. It has been estimated that, for humans, of the approximately 3 billion nucleotides which make up the genome, only about 3% are coding sequences. The shotgun and mapping approaches referred to supra do not differentiate between coding and non-coding regions. Hence, a method which would permit sequencing of only coding regions would be of great interest, especially if the method permits development of longer “contigs” of sequence information.

One such method is, in fact known. This is the “Expressed Sequence Tag” or “EST” approach. In this approach, one works with complementary DNA or “cDNA” rather than genomic DNA. In brief, as indicated supra, genomic DNA is transcribed into mRNA. The mRNA contains the relevant ORF in contiguous form, i.e. without intervening introns. These molecules are very fragile and their existence transient. In the laboratory, one can employ various enzymes, i.e., so-called “reverse transcriptases” to prepare complementary DNA, or “cDNA”, which is much more stable than mRNA. One then sequences the cDNA, incompletely, from either the 5′ or 3′ end. These incomplete sequences, in theory, serve as identifying “tags” for nucleic acid molecules of interest. Literally millions of ESTs have been prepared, and are accessible via known data bases, such as GenBank.

There are problems with this approach as well. First, large amounts of extremely high quality MRNA are necessary, and this is not always available. Also, one must bear in mind that the non-coding regions of mRNA molecules are found at the 5′ and 3′ ends, and this is carried over into the cDNA molecule. As a result, the information obtained may not be very useful. For example, it frequently provides no information about the actual protein encoded by the molecule. Clearly, there is a need for a system which provides more useful information about nucleic acid molecules.

Dias Neto et al., Gene 186: 135-142 (1997), the disclosure of which is incorporated by reference, applied a method for determining sequence information from the parasite S. mansoni which involved, inter alia, the use of arbitrary primers, and low stringency hybridization conditions. There is no discussion in this paper of the ability to identify and to sequence internal portions of an open reading frame. The paper itself appears to have only been cited a single time by other investigators. Nor is there any discussion within the reference of investigating sequences for overlap, so as to develop “contigs”, i.e, longernucleotide sequences prepared by determining overlap of two smaller sequences.

U.S. Pat. No. 5,487,985 to McClelland, et al., incorporated by reference, teaches a method referred to as “AP-PCR”, or arbitrarily primed polymerase chain reaction. The method employs a single primer designed so that there is a degree of internal mismatch between the primer and the template. Following amplification with the primer, a second PCR is carried out. The amplification products are separated on a gel to yield a so-called “fingerprint” of the organism or individual under study. The '985 patent does not discuss the identification of internal portions of open reading frames, nor does it discuss the analysis of sequences to develop contigs.

### BRIEF DESCRIPTION OF THE FIGURES

FIGS. 1A and 1B both show, schematically, prior art genome sequencing approaches.

FIG. 1C shows the invention, schematically.

FIG. 2 presents both a theoretical probability curve (dark ovals) and actual results (white ovals), obtained when practicing the invention. The data points refer to the probability of securing the sequence of a particular portion of cDNA molecule when practicing the invention.

FIG. 3 shows construction of a contig, using the invention.

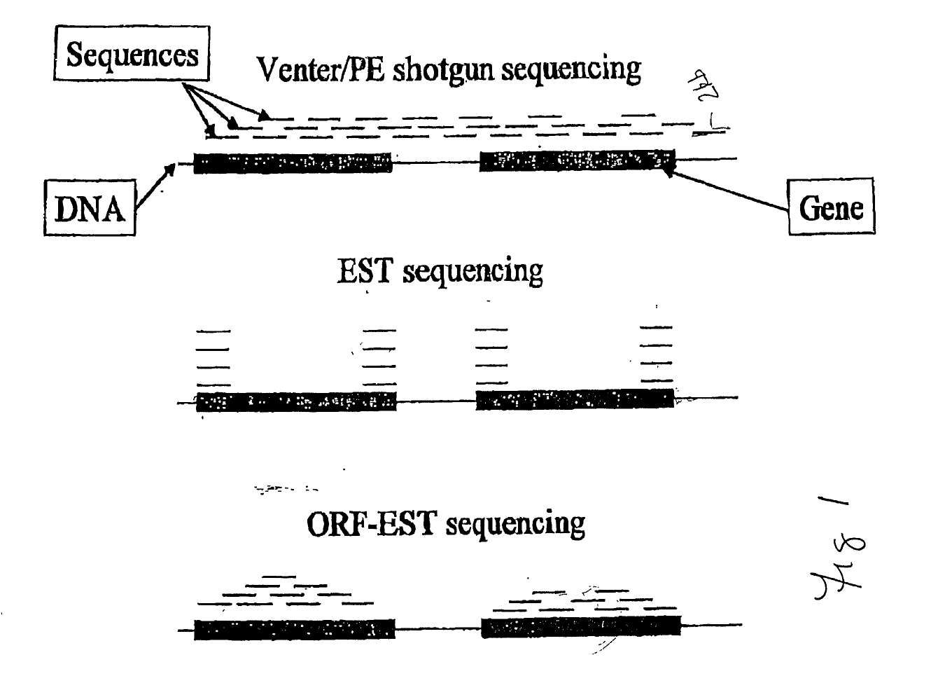

### DETAILED DESCRIPTION OF PREFERRED EMBODIMENTS

One aspect of the invention, as discussed supra, is a method for obtaining nucleotide sequence information from organisms, preferably information from open reading frames of cDNA of eukaryotic organisms. As a first step, messenger RNA (“mRNA”) is extracted from a cell. The extraction of MRNA is a standard technique, the details of which are well known by the artisan of ordinary skill. For example, it is well known that eukaryotic mRNA, as compared to other forms of RNA, is characterized by a “poly A” tail. One can separate MRNA from other types of RNA by passing it over a column which contains oligomers of the base thymidine. These “oligo dT” molecules hybridize to the poly A sequences on the mRNA molecules, and these then remain on the column. Other approaches to separation of mRNA are known. All can be used. If prokaryotic MRNA is being considered, separation using poly A/poly T hybridization is not carried out. It is preferred to treat the resulting material to reduce or to eliminate contamination by DNA. Adding a DNA degrading enzyme, such as DNA ase is preferred. This is carried out prior to contact with the column. It is also preferred to pas the purified RNA over the column at least twice.

The separated MRNA is then used to prepare a cDNA. The preparation of the cDNA represents the first inventive step in the method of the invention. To prepare the cDNA, the MRNA is combined with a sample of a single, arbitrary primer. By “arbitrary” is meant that the primer used does not have to be designed to correspond to any particular MRNA molecule. Indeed, it should not be, because the primer is going to be used to make all of the CDNA. Details on the design of arbitrary primers can be found in Dias-Neto, et al., supra, McClelland, et al., supra, and Serial No. 08/907,129 filed Aug. 6, 1997 and incorporated by reference.

The primer is preferably at least 15 nucleotides long. Theoretically, it should not exceed about 50 nucleotides, but it can. Most preferably, the primer is 15-30 nucleotides long. While the sequence ofthe primer can be totally arbitrary, it is preferred that the total content of nucleotides “G” and “C” in the primer be compatible with the “G” and “C” content of the open reading frames ofthe organism under consideration. It is found that this favors amplification of the desired sequences. General rules of primer construction favor a G and C content of at least 50%.

“Arbitrary primer” as used herein does not exclude specific design choices within the primers. For example, the four bases at the 3′ end of a given primer are generally considered the most important portion for hybridization. Hence, it is desirable to include as many different primers as possible, to cover all variations within this 4 base sequence. There are 256 variants possible, since there are four nucleotides. In order to identify products from a particular source, a “marker” sequence can be used, i.e., a stretch of predefined nucleotides. The remainder of the primer should be selected to correspond to overall GC usage, as described supra. Hence, for a primer 25 nucleotides long, the first 17 should correspond to GC usage for the organism in question. Nucleotides 18-21 would be a “tag”, such as “GGCC.” Then, all possible combinations of four nucleotides would follow, to produce 256 primers, which contain a known marker. This procedure could be repeated with a second set of primers, where the marker at 18-21 is different.

In practice, each set of variants is used with mRNA from a single source, and would permit the artisan to mark all sequences from a source, and still permit pooling.

The primer is combined with the MRNA under low stringency conditions. What is meant by this is that the conditions are selected so that the primer will hybridize to partially, rather than to only completely complementary sequences. Again, this is necessary because the primer will amplify an arbitrary sample of the MRNA pool, not just one sequence. There are standard rules and formulas for approximating high and low stringency, and the artisan of ordinary skill is familiar with these. Attention is drawn to Simpson, et al, U.S. Pat. application Ser. No: 08/907,129, filed Aug. 6, 1997, incorporated by reference, for more information on this, as well as Dias-Neto, et al. and McClelland, et al., supra.

The arbitrary primer and MRNA are mixed with appropriate reagents, such as reverse transcriptase, a buffer, and dNTPs, to yield a pool of single stranded, cDNA molecules.

Once the single stranded cDNA is prepared, it is used in an amplification reaction. In this second reaction, it is preferred, but not required, that the single primer used is identical to the first primer, as described supra, and that low stringency conditions be employed. Using identical primers tends to produce longer products, but this is not required.

The result of this amplification is a mini library. One can carry out cDNA synthesis in multiple, separate reactions, using different arbitrary primers, “A”, “B”, “C” and “D”. Four pools of single stranded cDNA are then produced, i.e, “A”, “B”, “C” and “D”. Each pool is then amplified using each of the four primers, to generate mini-libraries AA, AB, AC, AD, BA, BB, BC, BD, CA, CB, CC, CD, DA, DB, DC, and DD. These mini-libraries are used in the sequencing reaction which follows.

Once the cDNA is prepared, the resulting products are isolated, such as by size fractionation on a gel. The resulting bands can be removed from the gel, such as by elution, and then subjected to standard methodologies for cloning and sequencing.

Key to this feature ofthe invention, as is described herein, is the use of arbitrary primers under low stringency conditions. This combination permits the artisan to sequence internal regions of cDNA preferentially, as compared to the 5′ and 3′ ends, as is typical in standard prior art approaches. Specifically, consider a portion of a cDNA molecule which is a distance “S” from the 3′ end of the molecule. For this portion of the molecule to be amplified by a primer, the primer must bind on both sides of the region to be amplified. If the complete length of the molecule is represented by “L”, the probability of a primer binding to the nucleic acid molecule on both sides of a point on a nucleic acid molecule is S(L-S).

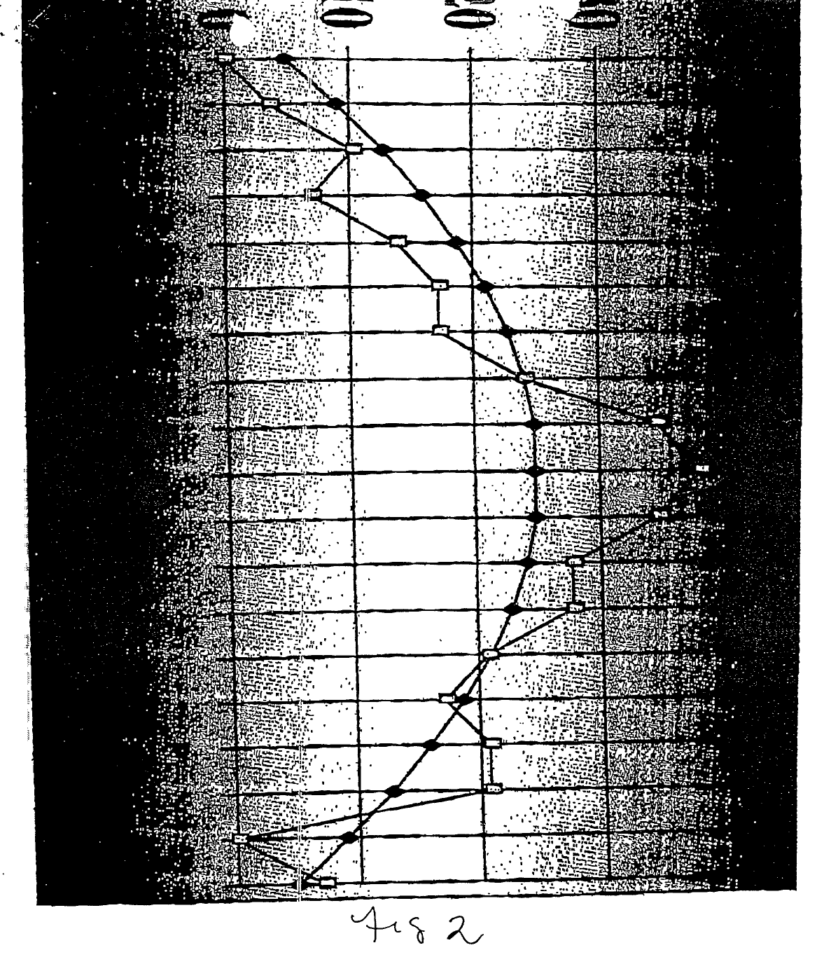

The highest probability for inclusion within amplified cDNA is the exact middle of the molecule. Lowest priority, in contrast, is at the extreme 5′ and 3′ ends. To elaborate, assume a point directly in the middle of a CDNA molecule, i.e., if the molecule is “+1” nucleotides long, .5x nucleotides precede the midpoint, and .5x nucleotides follow it. The likelihood of a primer hybridizing to a point on the molecule, preceding the middle is .5x, and following it is also .5x. If “x” is 1, then the probability of hybridization surrounding the midpoint is .5(1-.5), or .25, i.e., 25%. Similarly, assume a point on the same molecule located .9x away from the 3′ end. In this case, since the molecule is “x” units long, the point is .1x from the 5′ end, i.e., .1 units precede it, and .9 units follow it. If the length is 1, then the probability of hybridization surrounding this process is .9 (1-.9), or 9%. Hence, by using a primer and conditions which permit hybridization of the primer anywhere along the molecule, one actually secures the majority of amplified products from within a cDNA molecule, rather than at the ends. In FIG. 2 of this application, one sees a curve which results when the theoretical model is applied (dark ovals), and a curve obtained in practice (light ovals). It will be seen that, remarkably, the practice of the invention is actually very close to the theory.

One very practical result of this approach is that the mRNA is normalized, and bias in copy number is eliminated. The probability of producing an EST from a given mRNA is proportional to the length of that molecule and not its abundance within the source being analyzed.

A further aspect ofthe invention is the construction of contigs, once the sequence information has been determined. One creates a contig by comparing sequence information and finding overlaps. For example, the last 300 nucleotides of a sequence may be identical to the first 300 nucleotides of a second sequence. The artisan can essentially splice the first and second sequences together, to produce a longer one. The splicing can be done with two or more sequences found in the particular experiment that is carried out, or by comparing deduced sequences to sequences which are available in a public data base, a private data base, a journal, or any other source of sequence information.

A further aspect of the invention is the ability to compare information obtained using the inventive method to pre-existing information, in order to determine if a known nucleotide sequence is an internal sequence of a particular gene. This can be done because, as explained supra, the method described herein generates an extremely high percentage of internal sequences, with a very low percentage of sequences at the ends of a given molecule. The prior art methods either generate predominantly terminal sequences, or internal sequences on a completely random basis. Hence, it is probable that nucleotide sequences of unknown origin are contained within various sources of sequence information. Data generated using the methods of this invention can be compared to this pre-existing information very easily, and can result in a determination that a particular nucleotide sequence is, in fact, an internal sequence.

The practice of the invention and how it is achieved will be seen in the examples which follow.

### EXAMPLE 1

This example describes the generation of a cDNA library in accordance with the invention. While colon cancer cells from a human were used, any cell could also be treated in the manner described herein.

The mRNA was extracted from a sample of colon cancer cells, in accordance with standard methods well known to the artisan, and not repeated here. It was then divided into approximately 5μl aliquots, which contained anywhere from 1 to 10 ng of MRNA. The samples were then stored at - 70° C. until used.

The aliquots of MRNA were then used to prepare single stranded cDNA, using 25 pmol samples of a single, arbitrary primer. Several different experiments were carried out, using a different, single arbitrary primer in each case.

The single, arbitrary primers used were:

| | | |
|---|---|---|
| 5′-GAAGCTGGTA | AACAAAAGG-3′ | SEQ ID NO:392 |
| 5′-AGCTGCATGA | TGTGAGCAAG-3′ | SEQ ID NO:393 |
| 5′-CCCGCTCCTC | CTGAGCACCC-3′ | SEQ ID NO:394 |
| 5′-GAGTCGATTT | CAGGTTG-3′ | SEQ ID NO:395 |
| 5′-TGCTTAAGTT | CAGCGGG-3′ | SEQ ID NO:396 |

In each case, 25 pmols of arbitrary primer were mixed with the aliquot of MRNA, 100 units of Moloney murine leukemia virus reverse transcriptase, reverse transcriptase buffer (25mM Tris-HCl, pH 8.3, 75mm KC1, 3mM MgCl₂, 10 mM DTT), and 100 MM of each dNTP, to a final volume of 2OuL. The mixture was incubated for 30 minutes, at 37° C., to yield single stranded cDNA.

### EXAMPLE 2

The single stranded cDNA produced in example 1, supra, was used as the template in a PCR amplification reaction. In this, a sample of lul of single stranded cDNA was combined, together with the same primer that had been used to generate the cDNA. Amplification was carried out, using 12uM of primer, 200 uM of each dNTP, 1.5mM MgCl₂, 1 unit of DNA polymerase, and buffer (5OmM KC1, 10 mM Tris-HCl, pH9.0, and 0.1% Triton X-100), to reach a final volume of 15ul. Then, 35 cycles of amplification were carried out, 1 cycle consisting of 95° C. for 1 minute, (denaturation), 37° C. for 1 minute (annealing), and extension at 72° C., for 1 minute. In the final cycle extension was increased for 5 minutes. The amplification products were used in the analyses which follow. Additional experiments were also carried out, in the same fashion, using different primers.

### EXAMPLE 3

In order to analyze the amplification products, 3ul samples were mixed with 3ul of sample buffer, 0.05% bromophenol blue, 0.05% xylene cyanol FF, and 7% sucrose (w/v), in distilled water, and then visualized on silver stained, 6% polyacrylamide gels, following Sanguinetti, et al, Biotechniques 17:3-6 (1994), incorporated by reference.

The steps set forth supra result in banding patterns on the gel, each band representing a different sequence. The most complex banding patterns were analyzed, as discussed in example 4, infra. It is important to note that controls were run during the experiments, to make sure that genomic DNA had not contaminated the samples. In brief, the control experiments used mRNA and genomic DNA, without reverse transcription PCR. The profiles obtained should differ, in each case from those obtained using reverse transcribed mRNA, and did so.

### EXAMPLE 4

The cDNAs generated in the preceding examples were mixed, by pooling 10-20ul of each set of products into a final volume of 60ul, followed by electrophoresis through a 1% low melting point agarose gel containing ethidium bromide to stain the cDNA fragments. Known DNA size standards were also provided.

The gel portions containing fragments between 0.25 and 1.5 kilobases were excised, using a sterile razor blade. Excised agarose was then heated to 65° C. for 10 minutes, in 1/10 volume of NaOAc (3mM, pH 7.0), and cDNA was recovered via standard phenol/chloroform extraction and ethanol precipitation, followed by resuspension in 40ul ofwater. The thus recovered cDNA was used in the following experiments.

### EXAMPLE 5

The cDNA extracted supra was treated with 10 units of Klenow fragment cDNA polymerase, and 10 units of T4 polynucleotide kinase, for 45 minutes at 37° C. The reaction mixture was then extracted, once, with phenol, and the DNA was then recovered by passage through a standard Sephacryl S-200 column. Recovered cDNA was then ligated into the commercially available plasmid pUC18, and the plasmids were used to transform receptive E. coli, using standard methodologies. This resulted in sufficient amounts of individual cDNA molecules for the experiments which follow.

### EXAMPLE 6

Individual bacterial clones were established from the transformants of example 5. These were then used to prepare sequencing templates, following standard methodologies and sequenced. Standard computational procedures, and publicly accessible databases were employed in analyzing the resulting sequences. There were some cases where the analysis revealed two, different cDNAs in the clone. This could be determined, since the primer sequence is present only at both ends of the CDNA. Thus, if the primer was found in the middle of the sequence, it indicated that the sequences on either side were from different cDNAs. The two sequences were treated as separate sequences in analyzing the results.

Of 413 cDNA sequences studied, 337 were not found in the public databases referred to, supra. Sixteen of these sequences had a partial match to known sequences, allowing a contig to be formed.

There were another 42 sequences which were similar, but not identical to, sequences in public databases, suggesting that these 42 sequences are related to the pre-existing material.

Twenty six of the sequences were completely contained within known, complete human sequences. This permitted generation of the empirical curve shown in FIG. 2. Twenty two of the twenty six sequences were completely or partially within open reading frames of known genes.

Some of the sequences obtained showed partial homology to known genes, suggesting their function. Other sequences were found which showed no homology to known sequences.

Some of these sequences which were found in these experiments is set forth at SEQ ID NOS: 1 -241.

### EXAMPLE 7

This example shows the use of the invention as applied to breast cancer cells.

A sample of an infiltrative breast carcinoma with attached portions of normal tissues was operatively resected from a subject. The material was kept at -70° C. until used. The sample was characterized, inter alia, by a large tumor mass and a very small amount of normal tissue.

Three x 20 micron-thick slices were taken across the tumor mass and any attached normal tissue was microdissected out to leave “pure” tumor tissue. One slice was treated to remove MRNA, as described, supra. Three cDNA libraries were prepared, using SEQ ID Nos: 392 & 393, as well as

| | | |
|---|---|---|
| 5′-AGGAGTGACG | GTTGATCAGT-3′ | SEQ ID NO:397 |

Reverse transcription was carried out as with the colon cancer sample, as described supra. Then, PCR amplification was carried out by combining 12.8uM of the same primer used in the reverse transcription 125uM of each dNTP, 1.5 mM MgCl₂, 1 unit ofthermostable DNA polymerase, and buffer (5OmM KC1, lOmM Tris-HCl, pH 9.0, and 0.1% Triton X-100), to a final volume of20ul. Amplification was carried out by executing 1 cycle (denaturation at 94° C. for 1 minute, annealing at 37° C for 2 minutes, and extension at 72° C., for 2 minutes), followed by 34 cycles at 94° C for 45 seconds, annealing at 55° C. for 1 minute and extension at 72° C for 5 minutes. When analyzed for banding, as described supra, the samples revealed a complex pattern.

The products were eluted from their gels, cloned into pUC-18, and the plasmids were transformed into E. coli strain DH5a, all as described supra. Plasmids were subjected to minipreparation, using the known alkaline lysis method, and then about 150 of the molecules were sequenced. Of these, 69% were not found in any databank consulted, and appear to represent new sequences. A total of 22% was characterized by large quantities of repetitive elements and retroviral sequences. A total of 4% corresponded to known human sequences, another 4% to ribosomal RNA and mitochondrial sequences, and 8% were redundant sequences. The new sequences are set forth as SEQ ID NOS: 242-391.

### EXAMPLE 8

An example of how a contig sequence can be built is described herein.

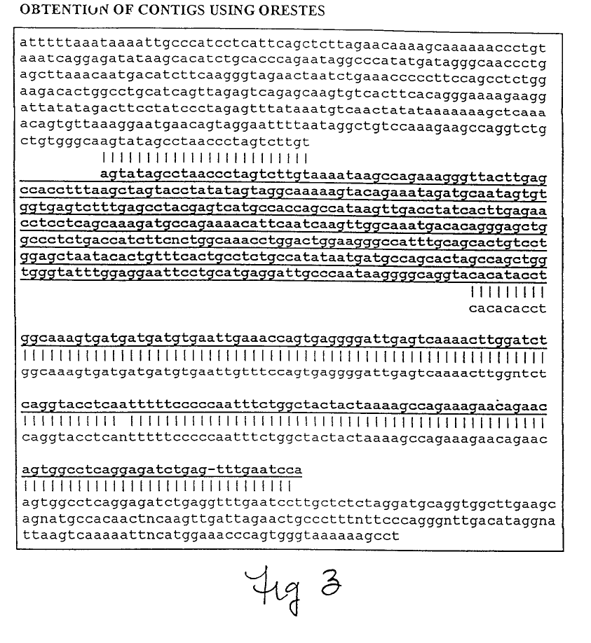

With reference to FIG. 3, the darker portion is a sequence obtained in accordance with the invention.

When the sequence was compared to sequences already accessible in databases, there was substantial overlap with a known sequence at the 3′ end, and some overlap at the 5′ end. This permitted construction of a 1,064 nucleotide long contig. The first sequence is a tentative human consensus sequence, as taught by Adams, et al, Nature 377: 3-17 (1995), while the third sequence is an EST obtained from human gall bladder cells, identified as human gall bladder EST 51121.

### EXAMPLE 9

The method described supra was used to screen a breast cancer library. The complete library of sequences obtained thereby are submitted herewith as the sequences which follow SEQ ID NO: 391 of the application.

The foregoing examples disclose the invention, one aspect of which is the identification of nucleotide sequences which correspond essentially in toto to coding regions or open reading frames of organisms. As shown, supra, the method involves forming a cDNA library by contacting a sample of mRNA with at least one arbitrary primer, at low stringency conditions, followed by reverse transcription. The resulting, single stranded cDNA is then amplified, with at least one arbitrary primer, at low stringency, to create a mini-library of cDNA. These nucleotide sequences are derived from internal, coding regions of mRNA. The resulting nucleic acid molecules are then sequenced. These can then be compared to a source of pre-existing sequence information, e.g., a nucleotide sequence library. Thus, pre-existing information which corresponds to internal MRNA sequences can be identified. Preferably, the method is applied to eukaryotes.

The method as described herein is applicable to any organism, including single cell organisms such as yeast, parasites such as Plasmodium, and multicellular organisms. All plants and animals, including humans, can be studied in accordance with the methods described herein.

More specific approaches using the inventive method will be clear to the skilled artisan. For example, one can determine sequences associated with cancer via, e.g., carrying out the invention on a sample of cancer cells and corresponding normal cells, and then studying the resulting mini-libraries for differences there between. These differences can include expression of genes in cancer cells not expressed in normal cells, lack of expression of genes in cancer cells which are expressed in normal cells, as well as mutations in the genes.

In another embodiment of the invention, one can determine if and where variation occurs in the nucleotide sequences of an organism. This can be done by producing sequences from different sources of an organism. These different sources can be, e.g., cells taken from different tissues, different individual organisms, and so forth. Such an approach will identify polymorphisms, among individuals and mutations present in specific pathological conditions, such as cancer. This approach can be accomplished using the “marked” primers as is described supra.

In addition to cancer, other pathological conditions can be studied. These conditions include not only mammalian conditions, such as diseases affecting humans, but also diseases of plants. Essentially, any scientific investigation which calls for analysis of a eukaryotic genome is facilitated by this aspect of the invention.

A second feature of the invention is a method for developing so-called “contig” sequences. These are nucleotide sequences which are generated following comparing sequences produced in accordance with this method to previously determined sequences, to determine if there is overlap. This is of interest because longer sequences are of great interest in that they define the target molecule with much greater accuracy. These contigs may be produced by comparing sequences developed in accordance with the method, as well as by comparing the sequences to pre-existing sequences in a databank. The aim is simply to find overlap between two sequences.

The power of the inventive method is such that there are innumerable applications. For example, it is frequently desirable to carry out analyses of populations of subjects. The invention can be used to carry out genetic analyses of large or small populations. Further, it can be used to study living systems to determine if, e.g., there have been genetic shifts which render an individual or population more or less likely to be afflicted with diseases such as cancer, to determine antibiotic resistance or non-tolerance, and so forth.

Studies on populations can also identify genes associated with diseases. Exemplary, but by no means inclusive of the types of conditions which can be studied are heart disease, bronchitis, Alzheimer's disease, diseases associated with particular human leukocyte antigens, autoimmune diseases, and so forth.

The invention can also be used in the study of congenital diseases, and the risk of affliction to a fetus, as well as the study of whether such conditions are likely to be passed to offspring via ova or sperm. Such analyses for pathological conditions can be carried out in all animals, plants, birds, fish, etc.

The invention, as discussed supra, is applicable to all eukaryotes, notjust humans, and not just animals. In the area of agriculture, for example, the genomes of food crops can be studied to determine if resistance genes are present, have been incorporated into a genome following transfection, and so forth. Defects in plant genomes can also be studied in this way. Similarly, the method permits the artisan to determine when pathogens which integrate into the genome, such as retroviruses and other integrating viruses, such as influenza virus, have undergone shifts or mutations, which may require different approaches to therapy. This aspect of the invention can also be applied to eukaryotic pathogens, such as trypanosomes, different types of Plasmodium, and so forth.

The method described herein can also be applied to DNA directly. More specifically, there are organisms, such as particular types of bacteria, which are very difficult to culture. One can apply the inventions described herein to DNA of these or other bacteria directly, rather than to cDNA prepared from MRNA. Essentially, the methodology used is the same as the methodology described supra, except genomic DNA is used. In such a case, random fragments are produced, rather than ORF segments. Using PCR in this type of approach means that very small amounts of DNA are needed, hence difficulties in culture are avoided. It is estimated that less than one microgram of DNA would be necessary to sequence an entire genome of a prokaryote.

Other aspects of the invention will be clear to the skilled artisan and need not be set forth herein.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

1. A method for determining open reading frames of the genome of an organism, comprising:
(a) contacting messanger RNA from a cell of said with a single, oligonucleotide primer at low stringency,
(b) preparing single stranded cDNA by reverse transcribing said messanger RNA with said dingle, oligonucleotide primer,
(c) amplifying said sing;estandard cDNA with a second, single oligonucleotide primer, to form an amplification product of nucleic acid molecules,
(d) sequencing the nucleic acid molecules of (c),
(e) repeating steps (a), (b) and (c) with a different pair of oligonucleotide primers, and
(f) sequencing nucleic acid molecules produced in (e).

2. The method of claim 1, wherin the oligonucleotide primers of (a) &(c) are identicle to each other.

3. The method of claim 1, wherin the oligonucleotide primers of (a) &(c) differ from each other

4. The method of claim 1, wherein said organism is a eukaryote.

5. The method of claim 4, wherein said eukaryote is an animal.

6. The method of claim 5, wherein said animal is a mammal.

7. The method of claim 4, wherein said eukaryote is a human.

8. The method of claim 4, wherein said organism suffers from pathological condition.

9. The method of claim 8, wherein said pathological condition is cancer.

10. The method of claim 9, wherein said cancer is colon cancer or breast cancer.

11. The method of claim 7, wherein said eukaryote is a multicellular organism.

12. The method of claim 4, wherein said eukaryote is not an animal.

13. The method of claim 12, wherein said eukaryote is a plant.

14. A method for determining that a known nucleotide sequence from a genome of an organism correspondes to a nucleotide sequence of an open reading frame, comprising:
(a) contacting messanger RNA from cell of said organism with at least one single stranded oligonucleotide primer, at low stringency,
(b) preparing single stranded cDNA by reverse transcribing said messanger RNA with said single, oligonucleotide primer,
(c) amplifying said single stranded cDNA with at least one, single stranded oligonucleotide primer, to form an amplification product, comprising of at least one nucleic acid molecule,
(d) sequencing said at least one nucleic acid molecule, and
(e) comparing the sequence determined in (d) to known nucleotide sequences for an organism for which said cell is taken to determine if any nucleotide sequences correspond to said at least one nucleic acid molecule, wherein any nucleotide sequences which do correspond are from an open reading frame.

15. The method of claim 14, wherein the olignucleotide primers of (b) and (c) are identicle to each other.

16. The method of claim 14, wherein the olignucleotide primers of (b) and (c) differ from each other.

17. The method of claim 14, wherein said cell is an eukaryote cell.

18. The method of claim 17, wherein said eukaryote cell is an animal cell.

19. The method of claim 18, wherein said animal is a mammal.

20. The method of claim 17, wherein said eukaryote cell is a human cell.

21. The method of claim 17, wherin said eukaryotic cell is associated with a pathological condition.

22. The method of claim 21, wherein said eukaryotic cell is a cancer cell.

23. The method of claim 22, wherein said cancer cell is a colon cancer cell or a breast cancer cell.

24. The method of claim 14, wherein said cell is a cell from a multicellular organism.

25. The method of claim 14, wherein said cell is a non-animal cell.

26. The method of claim 25, wherein said non-animal cell is a plant cell.

27. A method for preparing a contig, nucleic acid molecule from a ghenome of an organism, comprising:
(a) contacting messanger RNA from a cell with at least one oligonucleotide, at low stringency,
(b) preparing cDNA by reverse transcribing said messanger RNA with said single stranded oligonucleotide,
(c) amplifing said single stranded cDna with at lest one oligonucleotide primer to form an application product comprising at least one nucleic molecule,
(d) sequencing said at least one nucleic acid molecule,
(e) comparing the sequence of said at least one nucleic acid molecule to other nucleic acid molecules to determine any overlap there between, and
(f) constructing a contig nucleic acid molecule.

28. The method of claim 27, wherein said cell is an eukaryotic cell.

29. The method of claim 28, wherein said eukaryotic cell is an animal.

30. The method of claim 29, wherein said animal is a mammalian cell.

31. The method of claim 30, wherein said mammalian cell is a human cell.

32. The method of claim 28, wherein said eukaryouic cell is a plant cell.

33. The method of claim 27, comprising comparing said sequence and said at least one nucleic acid molecule electronically.

34. The method of claim 27, wherein the oligonucleotides of (a) & (c) are the same.

35. The method of claim 27, wherein the oligonucleotides of (a) & (c) differ from each other.

36. A method for sequencing all or part of a genome of an organism, comprising:
(a) contacting genomic DNA from a cell of said organism with a single oligonucleotide primer at low stringency, to generate a random set of nucleic acid molecules,
(b) amplifying said random set of nucleic acid molecules with a second oligonucleotide primer, to generate an amplification product,
(c) sequencing nucleic acid molecules in said amplification product,
(d) repeating steps (a), (b) and (c) with a different oligonucleotide primer, and
(e) sequencing nucleic acid molecules produced in (a).

37. The method of claim 36, wherein the oligonucleotide primers of (a) and (b) are identical to each other.

38. The method of claim 36, wherein said organism is a prokaryote.
